(19) United States
(12) Patent Application Publication
(10) Pub. No.: US 2003/0153077 A1
(43) Pub. Date: Aug. 14, 2003

(54) METHOD TO INCREASE THE RATE OF CELL GROWTH

(76) Inventors: William G. Pitt, Provo, UT (US); Steven Aaron Ross, Irving, TX (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/125,638

(22) Filed: Apr. 18, 2002

Related U.S. Application Data
(60) Provisional application No. 60/343,062, filed on Dec. 20, 2001.
(60) Provisional application No. 60/284,826, filed on Apr. 18, 2001.

(51) Int. Cl. C12N 13/00; C12N 5/02

(52) U.S. Cl. 435/383; 435/404; 435/173.1

(57) ABSTRACT

A method of increasing the growth rate of procaryotic and eucaryotic cells in culture is provided. The method teaches the exposure of the cells in culture to ultrasound of a selected frequency and intensity during incubation to improve cell growth. According to the methods of the invention, the frequency of the ultrasound may be from about 20 kHz to about 1 MHz, and the intensity of the ultrasound may be from about 1 mW/cm² to about 5 mW/cm².

### RELATED APPLICATIONS

This application claims the benefit of U.S. Provisional Application Serial No. 60/343,062, filed Dec. 20, 2001, entitled “Method to Increase the Rate of Cell Growth;” and U.S. Provisional Patent Application Serial No. 60/284,826, filed Apr. 18, 2001, entitled “Ultrasound Enhance Bacterial Growth.” Both of these applications are incorporated herein by reference.

### BACKGROUND OF THE INVENTION

1. Field of the Invention

The present invention relates to methods of increasing the growth rate of cultured cells. More specifically, the present invention relates to methods of increasing the rate of cell growth in a cell culture by exposing the cell culture to ultrasound.

2. Description of Related Art

Cell culture is a process that has found wide applicability in the sciences, medicine, pharmaceuticals, and even in industries such as food production. Methods of cell culture are also very widely applied to bacteria and other cells in laboratories all over the world for research purposes. In recent years, such methods have been increasingly utilized as recombinant DNA has all owed the production of medicinal proteins such as growth hormones and other regulatory factors in procaryotic organisms such as E. coli. Eucaryotic cells are also cultured and harvested to obtain hormones, proteins, and other biomolecules used in medicine or industry. In such industries, the production of the desired compound is dependent, at least in part, on the rate of growth of the cells which create it.

Medical applications of cell culture include the growth of human cells for replacement tissues. Replacement tissues of current interest are skin cells (for burn patients), chondrocytes (for cartilage replacement in joints, nose, ears, etc.), neurites and other nerve tissues (to replace or reconnect damaged nerves), cardiac tissues (for victims of heart attack or heart valve failure), endothelial cells (to line artificial or bioartificial blood vessels), liver and pancreatic cells (to replace diseased organs), and muscle cells (to replace damaged or lost muscle), among others. In nearly all of these examples of replacement tissue growth, the cells are harvested from a donor and seeded onto a substrate such as a set of degradable fibers and are initially grown in vitro without a blood supply to provide nourishment. This often limits the rate of growth of the cells, and results in tissues having a limited number of cell layers which require long growth periods. In many systems this is the case because poor nutrient penetration into the tissue limits the number of layers of cells that can be grown on the substrate.

In other technologies, yeasts, bacteria and other higher organisms are developed and cultured that have the ability to transform chemicals. One example of this is bacteria used in ethanol production from corn to provide an alternative fuel source to fossil fuels. Another example is bioremediation applications in which the organisms metabolize toxic chemicals into harmless substances. In these applications, the growth of the beneficial cells may be limited by poor nutrient distribution in their growth medium and the tendency of some cells to form layered films called “biofilms” on solid particles in soil or water which may be limited in thickness similar to the tissues mentioned above.

It is thus seen that providing methods of enhancing the growth rate of such cellular cultures would be a benefit in many arts, and could provide increased production of desired compounds, heightened ability to treat and cure disease and injury, and increased rates of resolution of harmful chemical spills.

Ultrasound is a technology that has found wide use in laboratories as well as in medicine. Ultrasound is commonly used in laboratories to clean solid surfaces such as the surfaces of glassware, metallic instruments, plastic parts, and more. For convenience, ultrasonic “cleaners” are built and sold to laboratories for such purposes. It is commonly believed that dust and particles are removed from these solid surfaces by cavitational events and related shear forces created adjacent to the surface by ultrasound. Specifically, it is thought that ultrasound waves may create cavitation bubbles in a liquid adjacent to a surface or in the liquid in the narrow volume between the surface and any loosely-attached “dirt” particles. The rapid expansion and contraction of these bubbles can cause extreme fluid shear forces that can knock particles from the surface. During transient cavitation the bubbles collapse adiabatically, causing extreme local temperatures, creating free radicals, and forming microjets. The latter are formed when the collapse of a bubble near a surface is distorted into a non-spherical collapse, and a high velocity jet of liquid impinges on the surface, again shearing particles from the surface. When transient cavitation occurs, the cleaning effect of ultrasound is significantly increased.

High intensity ultrasound has also been used to remove bacterial cells from solid surfaces. At very high power levels, most of the bacteria are removed. One group researched the possibility of applying ultrasound to one end of a pipe to strip bacteria from the lumen of the pipe. They quantified the removal of bacterial mass with infrared absorptiometry. They found that the ultrasound propagated axially with sufficient power to partially strip the bacteria from the entire length (50 cm) of the pipe. 87.5% of the bacteria were removed from 50 centimeter long tubes with frequencies around 100 kHz and intensities approaching 40 W/cm².

Another research group detached Pseudomonas diminuta biofilms from reverse osmosis membranes. They placed a point source of ultrasound at varying distances from a 1 cm² membrane. The power of the source was varied. The results indicated that even with power densities well above 2 Watts/cm², only 95% of the bacteria were removed. They attributed the detachment of the biofilm to transient cavitation.

Very high intensity (>10 W/cm²) ultrasound is also known to lyse bacterial and eucaryotic cells on surfaces and in suspension. This is the principle behind the cell “disrupter” commonly found in laboratories. Cavitational events are thought to break open or blow apart the cells, spilling their contents. Thus high intensity ultrasound can kill cells in addition to partially removing them from surfaces.

These uses of ultrasound have made it widespread and understood in research and industry communities. It would be beneficial in the art to provide additional methods for using such a popular technology.

Accordingly, a need exists for methods of enhancing the growth rate of cells including procaryotic and eucaryotic cells in culture. Further, a need exists for additional uses for ultrasound technology. A need also exists for methods of enhancing the growth of biofilms. Similar needs exist for methods that enhance the formation of tissues and the propagation of planktonic cell cultures. Such methods are taught herein.

### SUMMARY OF THE INVENTION

The methods of the present invention have been developed in response to the present state of the art. In particular, the methods have been developed in response to the problems and needs in the art not yet fully solved by currently available cell culture methods. The present invention provides a method of increasing the growth rate of cells in culture by exposing them to ultrasound.

In accordance with the invention as embodied and broadly described herein in the preferred embodiment, methods of increasing the growth rate of cells in culture are provided. Specifically, methods of cell culture are provided which include exposure of the cells in culture to ultrasound at specific frequencies and intensities. Exposure of these cells to this ultrasound does not destroy the cells. Instead, this exposure encourages the growth of the cells. This results in larger cell populations in the cultures exposed to ultrasound than in the cultures not exposed to ultrasound.

The methods of the invention use exposure to ultrasonic waves to enhance the growth of cells in culture. Cell growth appears to be enhanced by improved cellular access to nutrients in the medium. This happens because ultrasound increases the transport of small molecules in a liquid by increasing the convection present in the liquid. Increased convection in the liquid surrounding the cells enhances the growth of cells in several ways. Convection of the liquid increases the rate of small molecule flow, thus exposing needed nutrients to cells more rapidly. Further, convection reduces the depth of boundary layers that form near surfaces and that can prevent free flow of small molecules to the surface to which cells are often attached. The increase in convection appears to occur due to two different effects of ultrasound on the cell culture.

First, momentum from the ultrasonic waves can be transferred to the liquid, causing it to flow in the direction of the propagation of the sound waves. This phenomenon is termed “acoustic streaming.” Second, ultrasound forms microscopic gas bubbles in the liquid. These bubbles expand and contract with the low and high acoustic pressure waves of the ultrasound. This expansion and contraction causes shear flows around the bubbles, and is termed “cavitation.” Cavitation is considered “stable” when the acoustic pressure waves are not intense enough to completely collapse the bubbles when they contract. Very intense ultrasound can cause “collapse” cavitation, in which the radius of the bubble is reduced to zero during contraction. This collapse produces a shock wave, and the compression of the gas in the bubble generates high temperatures. Such temperatures may fragment molecules such as water, forming free radicals. Both stable and collapse cavitation increase convection in liquids. Collapse cavitation can kill cells, however.

Both stable and collapse cavitation are determined by both the acoustic frequency and the applied power density, also called the intensity. Cavitation is promoted at lower frequencies and higher intensities.

Thus, in the invention, cell cultures are exposed to ultrasound of a frequency and intensity sufficient to cause acoustic flow and/or cavitation. This promotes increased convection, and thus enhancement of cellular growth. It is preferable that the ultrasound produce primarily stable cavitation to avoid cell death caused by collapse cavitation. Some degree of collapse cavitation may be tolerated, however, so long as the net growth rate of the cells in the culture is positive.

Good results have been observed using the methods of the invention with both procaryotic and eucaryotic cells. Procaryotic cells are generally cells not having a membrane-enclosed nucleus, and often not having membrane-enclosed organelles. Examples of procaryotic cells include bacteria and cyanobacteria. Eucaryotic cells, on the other hand, are generally cells having membrane-enclosed organelles and nuclei. Examples of eucaryotic cells include most other forms of life, including plants and animals. Procaryotic and eucaryotic cells are both grown in laboratories for use in research. Both types of cells are also cultured in industry for applications including the production of useful cellular products such as therapeutic proteins or hormones.

Cell culture is a term describing methods for encouraging and/or allowing cells to live, grow, and multiply in conditions set up and controlled in a laboratory environment. This generally involves growing a cell or group of cells in a culture dish or flask. The dish or flask generally contains a measure of a growth medium that contains nutrients required for cell growth. Cells are placed on/in the media, and are incubated by exposing them to conditions of light, temperature, etc., that encourage growth, and often, division, of the cells. The composition of the growth medium and the conditions of light, temperature, etc., that encourage growth and division may be varied to suit the needs and requirements of the cells to be cultured.

Good results have similarly been observed when using the methods of the invention with cells cultured as biofilms and with cells cultured as planktonic suspensions. A biofilm is a collection of cells and their exudates attached to or associated with a substrate, thus being a film on a substrate containing living cells. Planktonic cell cultures are those cell cultures in which the cells are grown suspended in and often distributed throughout the media.

In the methods of the invention, the frequency of the ultrasound used may be chosen from a range of about 20 kHz to about 1 MHz. At these frequencies, under appropriate levels of acoustic intensity, stable cavitation is created, thus enhancing convection and cell growth. The range may also include some frequencies and intensities at which some collapse cavitation is present. At such frequencies, some cell death may be observed. The frequency is useful in the methods of the invention, however, so long as cell growth rates show a net positive cell growth. In some situations, and under some conditions, the frequency of the ultrasound used in the invention may also be from about 20 kHz to about 100 kHz. At these lower frequencies, there is more cavitation present, which cavitation enhances convection. At low intensities at these low frequencies, stable cavitation predominates over collapse cavitation.

In the methods of the invention, the intensity of the ultrasound may be from about 1 mW/cm² to about 5 W/cm². At these intensities, under most conditions, stable cavitation is created, thus enhancing convection and cell growth. The range may also include some intensities at which some collapse cavitation is present. At such intensities, some cell death may be observed. The intensity is useful in methods of the invention, however, so long as cell growth rates show a net positive cell growth.

In some situations, especially in applications directed to procaryotic cells, the intensity of the ultrasound used in the invention may also be from about 1 W/cm² to about 5 W/cm². At these intensities, both stable and collapse cavitation are present; however, procaryotic cells are more resilient to collapse cavitation than eucaryotic cells, and they can survive in the presence of a small amount of collapse cavitation. The range of 1 W/cm² to about 3 W/cm² may also be useful in order to provide cavitation and enhanced convection. These higher ranges of ultrasound intensity are more suitable for use with procaryotic cells.

In other applications, such as those directed to eucaryotic cells, the intensity may be from about 1 mW/cm² to about 50 mW/cm². At these lower intensity ranges, convection is still enhanced by stable cavitation, while levels of collapse cavitation are very low. The intensity range of from about 8 mW/cm² to about 18 mW/cm² may also be useful in order to provide stable cavitation and enhanced convection. Methods including such intensity ranges are better suited for the culture of eucaryotic cells. Eucaryotic cells are generally more vulnerable to damage from collapse cavitation.

A first set of the methods of the invention are for increasing the growth rate of a procaryotic cell in culture. In these methods, the cell is exposed to ultrasound of a frequency of from about 20 kHz to about 1 MHz, and of an intensity of from about 1 mW/cm² to about 5 W/cm². As briefly noted above, procaryotic cells are more resilient than eucaryotic cells, and can thus often withstand the higher frequencies and intensities present in these ranges, as well as some collapse cavitation. In alternate methods, the procaryotic cell is exposed to ultrasound having a frequency of from about 20 kHz to about 100 kHz, and an intensity of from about 1.5 W/cm² to about 2.5 W/cm². This range may be more preferred for use with procaryotic cells more resistant to high intensities and collapse cavitation. In both of these ranges, procaryotic cells showed increased cell growth following exposure to ultrasound during incubation.

A next set of the methods of the invention are for increasing the growth rate of a eucaryotic cell in culture. In these methods, the cell is exposed to ultrasound of a frequency of from about 20 kHz to about 1 MHz, and of an intensity of from about 1 mW/cm² to about 1 W/cm². As explained above, eucaryotic cells are often disrupted or damaged by higher ranges, and thus cannot tolerate lower ranges of frequency and high intensity. In alternate methods, the eucaryotic cell is exposed to ultrasound having a frequency of from about 20 kHz to about 100 kHz, and an intensity of from about 8 mW/cm² to about 50 mW/cm². This intermediate range may be preferred for eucaryotic cells because convection from stable cavitation is increased while collapse cavitation is absent or minimal. In both of these sets of frequency and intensity ranges, eucaryotic cells showed increased growth during exposure to ultrasound.

These and other features and advantages of the present invention will become more fully apparent from the following description and appended claims, or may be learned by the practice of the invention as set forth hereinafter.

### BRIEF DESCRIPTION OF THE DRAWINGS

In order that the above-recited and other features and advantages of the invention will be readily understood, a more particular description of the invention is given. Specific examples thereof are detailed, the results of which are illustrated in the appended figures. The following examples are only typical embodiments of the invention, and are not to be considered to limit its scope. In the accompanying figures:

FIG. 1 is a chart showing approximate ranges of frequency and intensity of ultrasound at which the onsets of stable and collapse cavitation have been observed. The upper left quadrant represents a region wherein both types of cavitation are present in water, while the lower right corner represents a region of no cavitation;

FIG. 2 is a chart showing the amounts of bacteria adherent to polyethylene rods after one hour exposure to 10⁵ CFU/ml S. epidermidis as a function of the intensity of 70 kHz ultrasound;

FIG. 3 shows two sets of stained S. epidermidis biofilms adhered to polyethylene rods, the biofilms having been grown for 16 hours with and without the presence of 2 W/cm² ultrasound and stained with toluidine blue. Rods incubated with exposure to ultrasound are shown in the left of each photo;

FIG. 4 shows the absorbance of stained 48-hour P. aeruginosa biofilms extracted from polyethylene rods that had been exposed to 1:5 pulsed 2.2 W/cm² 70 kHz ultrasound, 1:5 pulsed 1.5 W/cm² 70 kHz ultrasound, and no ultrasound;

FIG. 5 shows the growth of planktonic S. epidermidis with 70 kHz ultrasound at 3 W/cm² (circles) and without ultrasound (triangles); with the data shown being the mean and 95% confidence intervals of 4 replicates; and

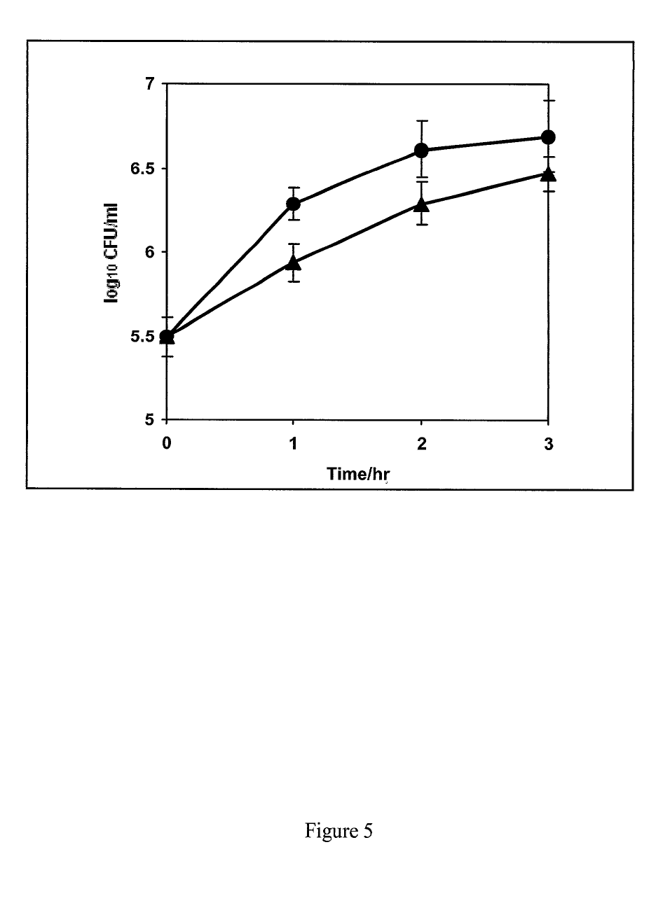

FIG. 6 shows the results of Example 6 in which E. coli biofilm formation was enhanced by exposure to ultrasound. The blue rings on each rod are not biofilm, but are stained dried tryptic soy broth that dried at the interface between the air and the bacterial culture during the experiment. The biofilm occupies the area between the blue rings and the end of the rod. The rods on the left are the insonated rods, and the rods on the right were not exposed to ultrasound.

### DETAILED DESCRIPTION OF THE PREFERRED EMBODIMENTS

The presently preferred methods of the invention will be better understood by referring to the following examples with their attached figures. The methods of the present invention, as generally described herein, can be practiced and varied in many ways. Thus, the following more detailed description of the methods of the present invention is not intended to limit the scope of the invention, as claimed. Instead, the detailed description is merely representative of presently preferred embodiments of the invention.

### Definitions

As used herein, the term “biofilm” is used to connote a collection of cells, including both procaryotic and eucaryotic cells, and their exudates such as exopolysaccharides and other exuded products, attached to or associated with a substrate. These cells thus form a film on a substrate containing living cells.

The term “substrate” may include a solid or porous substance such as membranes, meshes (including woven and non-woven meshes), and filters, on which cells may grow. Such substrates may be of many compositions, including polymeric compositions.

“Ultrasound” is used to describe generally acoustic energy and acoustic waves capable of producing stable or transient cavitation or enhanced convection in a medium by cavitation or acoustic streaming. As such, ultrasound includes, but is not necessarily limited to, frequencies greater than about 20 kHz. The terms “insonation” and “ultrasonication” and their derivatives are descriptive of the process of exposing something to ultrasound for any period of time at any frequency or intensity.

“Planktonic” cellular cultures include those cultures capable of growth without being anchored to a substrate. Such cultures are capable of growth in suspension in a medium such as those used herein or their equivalents.

### Discussion

Cells are cultured for many purposes. Applications for cultured cells include, but are not limited to, research; the production of pharmaceuticals, chemicals, proteins or other therapeutic polypeptides, hormones, or other useful biomolecules; diagnostic devices or techniques that require cell growth as part of an assay; and increasingly to produce replacement tissues. The present invention relates to the use of ultrasound to increase the rate of growth of cells in culture. Without being limited to any one theory, it appears that the application of ultrasound to cells results in an increase in the transport of nutrients and oxygen to the cells. Ultrasound is used judiciously so as to avoid killing the cells on a substrate or in suspension. In some applications, care is used to avoid removal of cells from a substrate.

As noted above, it is thought that ultrasound increases the transport of small molecules in a liquid solution. It does so by increasing the convection present in an otherwise stagnant or relatively slower moving fluid. There is generally a boundary layer of stagnant fluid located adjacent to surfaces which creates a resistance to the transport of small molecules to the surface. Increased convection in the bulk fluid reduces the thickness of this boundary layer and increases transport to the surface. It is desirable to increase the transport of oxygen and nutrients to cells growing on a surface. It is also desirable to speed the transport of cellular waste products away from the cells on the surface in order to increase the growth rate of the cells.

Ultrasound increases convection in a liquid by at least two mechanisms. The first is acoustic streaming in which momentum from directed propagating sound waves is transferred to the liquid, causing the liquid to flow (albeit slowly) in the direction of the sound propagation. Acoustic streaming happens to varying degrees at any intensity of audio sound or ultrasound. The second mechanism is microconvection created by cavitating gas bubbles formed in the liquid by the ultrasound.

The cycles of low and high acoustic pressure created by the ultrasound cause the gas bubbles to expand and shrink, which in turn creates shear flow around the oscillating bubbles. Stable cavitation results when the acoustic intensity is sufficiently low that the bubbles do not collapse completely during their contraction cycle. The onset of stable cavitation greatly increases convective transport, and such transport increases with increasing acoustic intensity as larger and more numerous cavitation bubbles form. As the acoustic intensity continues to increase, convection increases.

Convection jumps dramatically when collapse (or “transient”) cavitation begins to occur. Collapse or transient cavitation is present when the intensity of the acoustic pressure waves drives the radius of the cavitating bubble to zero during the contraction cycle. The sudden collapse produces a shock wave. The adiabatic compression of the gas produces temperatures on the order of 5000 K. During collapse cavitation, convection is greatly enhanced, and the local temperatures can fragment water and other molecules, thus forming free radicals.

It was found that ultrasound of ranges of specific frequencies and intensities allowed the adhesion of procaryotic cells to substrates. Following this, it was observed that low frequency ultrasound of low acoustic intensity increased the growth of such cells compared to those allowed to grow without ultrasound. Similar observations were then made in regard to eucaryotic cells in planktonic culture and biofilms. It appears that ultrasound increases the rate of transport of oxygen and nutrients to the cells and increases the rate of transport of waste products away from the cells, thus enhancing their rate of growth.

The invention thus includes the disclosure of a window of frequencies and intensities of ultrasound which increase the net rate of cell growth. Cell removal from a biofilm and cell death are not generally desired within the invention, although some degree of cell removal or cell death may be allowed as long as the enhanced rate of cell growth is greater than the rate of cell removal. The invention also includes the determination that cells can successfully attach to a surface during the application of ultrasound, thus enabling “cell seeding” to occur at the same time as enhanced nutrient transport.

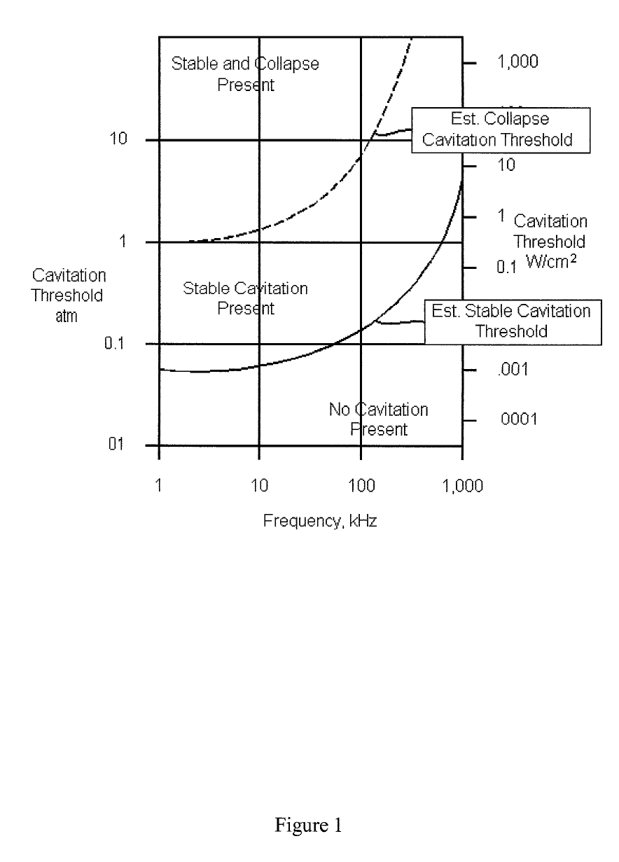

Since ultrasound is commonly used to remove bacteria from surfaces, a common misconception is that ultrasound might also be expected to prevent biofilm formation and even to reduce bacterial growth. It appears that there is an intensity window between promoting cell growth and killing cells. Procaryotic cells are generally best suited to culture at the higher end of this intensity window, and eucaryotic cells are generally best suited to culture at the lower end of the window. As portrayed in FIG. 1, this window is between about 1 mW/cm² and about 100 W/cm² at 20 kHz. At 70 kHz, both the intensity at which collapse cavitation begins to occur (represented approximately by the upper line) and the intensity at which stable cavitation begins to occur (represented approximately by the lower line) increase. Below the threshold of stable cavitation, no cavitation occurs, and the very little benefit experienced by the cells in culture is derived only from acoustic streaming.

FIG. 1 represents that cavitation and the subsequent increase in convective transport is not limited to the region of ultrasound of 20 kHz or higher, as commonly defined by physicists. Cavitation extends into the range of acoustic sound, and thus enhanced cell growth is expected to be found in that region also.

In regard to the ultrasound enhancement of biofilm formation, it has been taught in the art that a nutrient concentration gradient exists within biofilms. The mass-transfer resistance of the exopolymers in the biofilm slows the bacterial growth rate. Using mathematical models, ultrasound has been shown to increase the permeability of biofilms to small molecules. Ultrasound thus functions to increase the convection of small molecules including nutrients and to increase the permeability of biofilms to allow better absorption and subsequent cell growth.

Examples of the enhancement of biofilm growth include observation of enhanced growth in cultures of S. epidermidis, E. coli and P. aeruginosa. Ultrasound increased the biofiln growth more in the S. epidermidis bacteria than in the other two species. Further, ultrasound increased the biofilm growth more in the E. coli experiments than the P. aeruginosa experiments. These differences are likely due to the natural differences in the metabolic growth rates of the bacteria without ultrasound. In biofilms grown without ultrasound, the growth rate of biofilms without ultrasound decreases in the order of S. epidermidis>E. coli>P. aeruginosa. All three species demonstrated an increase in biofilm formation while exposed to 2 W/cm² ultrasound.

With respect to increased growth of eucaryotic cells, ultrasound also increases the transport of nutrients and oxygen to these cells in the same way as with procaryotic cells. In general, eucaryotic cells are much more sensitive to their environment than procaryotic cells, and they do not grow, and often will die, when the correct levels of oxygen and nutrients are missing. The transport of nutrients and oxygen into a growing cell layer in vitro is the limiting factor with respect to how thick the cell layers will grow. Therefore the increase in convective transport associated with exposure to ultrasound leads to faster growth of these cells. Ultrasound has also been shown to increase the uptake of extracellular molecules by increasing the permeability of the cell membrane.

Mammalian cells lack the rigid cell wall that gives structure and strength to bacterial cells. Therefore they are more prone to be lysed by ultrasonic exposure. Experiments in which eucaryotic cells were cultured used ultrasound of an intensity much lower than the intensity used in experiments conducted with procaryotic cell cultures.

The application of ultrasound to enhance the growth of cells potentially has numerous applications and significant beneficial potential. One such benefit is the more rapid growth of bacteria and other cells in the lab for research purposes. Another could be the production of pharmaceuticals, replacement cells, and tissues for transplant from cell culture. Currently the bacterium E. coli has been engineered to contain recombinant DNA to produce medicinal proteins such as growth hormones and other regulatory factors. Eucaryotic cells are also cultured and harvested to obtain hormones, proteins, and other biomolecules used in medicine or industry. Increased rates of growth could potentially increase the production of desired compounds as well as lower the costs of such naturally-produced biomolecules.

Another potential medical application of this technology is the growth of human cells for replacement tissues. Replacement tissues of current interest are skin cells (for burn patients), chondrocytes (for cartilage replacement in joints, nose, ears, etc.), neurites and other nerve tissues (to replace or reconnect damages nerves), cardiac tissues (for victims of heart attack or heart valve failure), endothelial cells (to line artificial or bioartificial blood vessels), liver and pancreatic cells (to replace diseased organs), muscle cells (to replace damaged or lost muscle), and more. In nearly all these examples of replacement tissue growth, the cells are harvested from the donor and seeded onto a substrate such as degradable fibers. Cells are initially grown in vitro without a blood supply to provide nourishment. Ultrasound can increase the diffusion of nutrients and oxygen into the cell masses, allowing tissue cultures to be grown thicker and faster. In many systems the nutrient penetration into the tissue is the limiting factor in the number of layers of cells that can be grown on a surface. Therefore ultrasound can be used to enhance this transport and increase the growth rate of these tissues, allowing burn patients, accident victims, and heart attack patients, and even children with birth defects to be healed faster.

Another application is in biocultures of yeasts, bacteria and other higher organisms that transform chemicals. Such an example is ethanol production from corn to provide a fuel source. Another example is in bioremediation in which the organisms metabolize toxic chemicals into harmless substances. In bioremediation the bacteria are often found in biofilms on solid particles in soil or water. Their enhanced growth via ultrasound would increase the rate of clean up of these chemicals.

In the invention, the methods of increasing the growth rate of procaryotic and eucaryotic cells in culture utilize ultrasound of frequencies and intensities of specific ranges. Specifically, ultrasound having a frequency of from about 20 kHz to about 1 MHz is generally preferred in the practice of the invention to generate stable cavitation. Under conditions when more cavitation is desired, ultrasound having a frequency of from about 20 kHz to about 100 kHz is preferred. In some circumstances, ultrasound having a frequency of 70 kHz is preferred to create cavitation and increased convection.

Further, the intensity of the ultrasound is selected from a range of from about 1 mW/cm² to about 5 W/cm². Throughout this range of intensities, stable cavitation is generally created, and collapse cavitation is absent at the lower intensities, but becomes significant at the higher intensities. Higher intensities are preferred for use in methods of culture of procaryotic cells, and lower intensities are preferred for use in methods of culture of eucaryotic cells. In methods of culture of procaryotic cells, ultrasound having an intensity of from about 1 W/cm² to about 3 W/cm² may be preferred when more vigorous cavitation and subsequent convection is preferred, or when less resistant procaryotic cells are being cultured. For an optimum amount of cavitation, producing a compromise between cell growth and cell death by collapse cavitation, ultrasound having an intensity of from about 2.0 W/cm² to about 2.2 W/cm² may be used. When culturing eucaryotic cells, intensities of about 1 mW/cm² to about 50 mW/cm² may be preferred to prevent damage or cell death and to instead encourage growth. Further, in intensities of from about 8 mW/cm² to about 18 mW/cm² may be preferred to provide stable cavitation to eucaryotic cells less resistant to damage from cavitation.

As mentioned above, these methods are suited for use with cells grown in suspension such as planktonic cultures, as well as with cells grown on a substrate, including biofilms.

These examples are meant to be exemplary and not to in any way limit the possible uses of ultrasound in enhancing cell growth, whether the cells are procaryotic or eucaryotic, whether the cells are found on surfaces or not.

### EXAMPLES

### Materials and Methods

A. Procaryotic Species & Methods

Although it is anticipated that the methods of the invention are suitable for use with many procaryotic species, three species of bacteria were used in the development of the procaryotic portion of this invention. These are known to colonize surfaces. Staphylococcus epidermidis (strain RP62A, ATCC #35984), Pseudomonas aeruginosa (ATCC #27853) and Escherichia coli (ATCC #10798) were used in the examples of the invention discussed below. The bacteria were stored as frozen cultures and inoculated onto nutrient plates weekly. Tryptic soy broth (“TSB”) was inoculated with one colony from the plate, and a culture was grown overnight at 37° C. with shaking. In some of the examples involving the growth of S. epidermidis, 0.25 wt % glucose was added to the TSB.

B. Materials

Polymer rods were selected for this study as an exemplary material for the adhesion and growth of bacteria on a surface. Though many materials of varying shapes and configurations are likely suitable for the practice of the invention, the following examples were performed with rods made of high density polyethylene. The rods used had a diameter of 0.12 cm and were approximately 15 cm long. In test tubes filled with 2 ml of TSB, the bottom of the rod along a length of 0.801 cm was exposed to the bacterial suspension and/or nutrient broth. This length corresponded to an overall exposed area of about 0.313 cm².

Prior to use, new rods were prepared by cleaning them with ethanol in an ultrasonic bath. Additionally, before the rods were used in each example, they were sterilized in an autoclave for twenty minutes. When rods were reused, the rod surfaces were first scrubbed with soap and then processed in ethanol in the ultrasonic bath after each use and then autoclaved just prior to the next experiment.

Toluidine blue, a standard histology stain, was used to stain both the bacteria and their exopolysaccharides and other cell exudates, thus making them better visible for observation. Toluidine blue absorbs light at 590 nm. A literature report previously determined that using toluidine blue to stain the biofilm provided the most reproducible procedure. Since toluidine blue stains the polysaccharides and the bacterial cells of the biofilm, measurements of total absorbency obtained from staining experiments correlate with total biofilm volume.

The experiments required known intensities of ultrasound to be applied to the polyethylene surfaces while the surface was exposed to the bacteria. Sonicor SC100 ultrasonic baths (Copiaque, N.Y.) operating at 70 kHz were used as the source of ultrasound. A test tube rack was placed inside the bath to support glass test tubes containing the rods, and a Bruel and Kjaer hydrophone (Model 8103, Naerum, DK) inside a test tube was used to quantify the intensity applied to each location within the rack in the ultrasonic bath. The ultrasonic intensity inside a test tube was measured before and after each experiment. Acoustic intensity was varied by changing the AC voltage to the ultrasound bath as described previously.

C. Procedures

In a first example of the invention, the initial adhesion of bacteria to the rods was measured according to the following procedure. A 24-hour S. epidermidis culture was diluted 1 to 1000 into fresh TSB. The diluted culture was incubated at 37° C. for 4 hours. During this time, the cells grew to a concentration of about 10⁵ cells per milliliter. At this point, 2 ml of cell culture was pipetted into each of 8 test tubes. A polyethylene rod prepared as discussed above was placed into each test tube. The 2 ml of culture in the test tube covered about 0.313 cm² of the bottom end of the rod.

Four of the above tubes were placed into a bath exposed to ultrasound at 37° C. at specified power densities. The remaining 4 tubes were placed into a 37° C. incubator on a shaker set at 70 rpm. The rods were exposed to the culture for 1 hour, after which the rods were rinsed in physiological saline solution (“PSS”) to remove non-adherent bacteria.

The relative amounts of bacteria adherent to the rods were then assessed by stripping and plate counting. A standard procedure for stripping the bacteria from the rod surfaces followed by plate counting was used in these experiments. The procedure calls for the use of ultrasound at higher power densities (2-4 W/cm²) under conditions commonly understood to remove bacteria, though it is understood that the procedure probably does not remove 100% of the bacteria. The procedure does, however, remove a consistent percentage of the bacteria, and can thus be used to compare the relative amounts of bacteria adherent under different conditions.

First, each rod was rinsed with three two-milliliter squirts of PSS to remove planktonic bacteria. Each rod was then placed into another test tube filled with 2 ml of PSS. The test tubes were placed into an ultrasonic bath with power densities set for stripping powers (2-4 W/cm²). The test tubes were allowed to stay in the ultrasonic bath for 30 minutes. The rods were removed and the bacterial concentration of the remaining suspension was measured by standard plate counting techniques. In these techniques, the suspension was serially diluted and plated on nutrient agar plates. Colonies were counted after 48 hrs of incubation at 37° C.

The presence of bacteria and their associated exopolysaccharides in a biofilm on the rods was measured by toluidine blue. In staining the biofilms, the procedure described below was followed. After the rods were exposed to the bacteria they were rinsed by dipping them into a test tube containing 2 ml of PSS. Each rod was then placed in 2 ml of Camoy's solution (60% ethanol, 30% CHCl₃, 10% glacial acetic acid) for 10 minutes. Each rod was next placed in 2 ml of 1% toluidine blue stain for 1 hour. Following this, each rod was briefly rinsed in a test tube containing 2 ml of PSS, and then placed into 1 ml of 0.2 M NaOH at 80° C. for 1 hour. Finally, the rods were removed and the absorbance of the remaining solution was measured at 590 nm in a spectrophotometer.

The absorbance of a clean rod subjected to the above procedure was used as a control. The difference between the absorbance obtained from a test rod and the control rod was considered proportional to the amount of cells and exopolysaccharide on the test rod. In some experiments with S. epidermidis and E. coli, the biofilm was sufficiently dense that it could not be removed from the rod by the NaOH digestion above. In these cases, the blue stain remained on the rod and was photographed.

Planktonic suspensions: an overnight culture of bacteria in TSB was diluted 1:1000 in fresh TSB and grown at 37° C. for 2 hours (S. epidermidis) or 3 hours (E. coli and P. aeruginosa). The culture was separated into individual test tubes containing 2 ml growing culture. Half of these tubes were placed in the bath exposed to ultrasound, and the other half were incubated without ultrasound. At regular time intervals, samples were withdrawn, serially diluted, and plate counted.

D. Eucaryotic Species & Methods

Three species of mammalian cell lines were used to evaluate the utility of the methods of the invention in relation to eucaryotic cells. HeLa and WiDR are species that grow well on surfaces, and are thus designated anchorage dependent. TK-6 cell lines grow in suspension, and are thus designated anchorage independent. All of these cells were grown at 37° C. in RPMI media supplemented with glutamate, gentamicin, and 10% calf serum.

E. Procedures

Two identical Sonicor ultrasonic baths were cleaned and placed in a cell incubator maintained at 37° C. with 5% CO₂. Only one bath was powered. A recirculating pump continuously circulated water from one bath to the other so that they were always kept at the same temperature. The power to the operating bath was adjusted by plugging the bath into a variable AC transformer supplied with 120 V AC. The acoustic intensity experienced by the cell in the bath was calibrated with a Bruel & Kjaer hydrophone that was placed through a hole cut in the top of a 25 cm² tissue culture flask (T25 flask) that was filled with 7 ml of water and that was floating on the surface of the water in the Sonicor. The ultrasonic power density in the liquid in the flask was correlated with the amount of voltage supplied by a variable voltage transformer. During the eucaryotic experiments detailed in the Examples section below, two flasks were floated in each Sonicor bath.

Supplies of cells were maintained in T25 or T75 flasks using standard cell culture techniques. To perform experiments with HeLa or WiDR cells, the media was first aspirated from a supply flask. Four ml of trypsin solution was next added to it and allowed to remain for 1 minute. Following this, 3 ml of the trypsin was removed and the flask was placed in the incubator for about 10 minutes, or until all of the cells could slide loose from the flask and the large clumps were mostly separated. Following this, 3 ml of media was added and the flask was swirled to properly distribute the media around the cells. A sample of the resulting suspension was taken, and a count was taken of the total number of cells and of the number of viable cells. This count was conducted using the trypan blue technique.

The cell suspension was then diluted to the desired cell concentration. This concentration generally ranged from about 0.2×10⁵ to 0.8×10⁵ cells/ml. Then 7 ml of this suspension was pipetted into each of 4 flasks. In most experiments, flasks with filter caps were used. The cells were incubated for 24 hrs before two of the flasks were exposed to ultrasound for 24 or 72 hours.

When using TK-6 cells, the cells were first pipetted from a tissue culture flask into a sterile flask, where the cell suspension was diluted with fresh media to a concentration of about 0.71×10⁵ cells/ml. Then 7 ml of this suspension was pipetted into each of 4 flasks. The cells were incubated for 24 hours. After this, two of the flasks were exposed to ultrasound for 24 hours.

After simple incubation or incubation with ultrasonic exposure, the cells were counted using the trypan blue procedure. The cell suspension was pipetted from the flask and the cell concentration was counted. The amount of cells adherent to surfaces of the flask was determined by adding four ml of trypsin solution for 1 minute. Following this, 3 ml of the trypsin were removed and the flask was placed in the incubator for about 10 minutes, or until the cells could slide free from the back of the flask and the large clumps of cells were mostly separated. Next, 3 ml of media was added and the flask was swirled to distribute the medium around the cells. A sample of the resulting suspension was taken, and a count was made of the total number of cells and the number of viable cells in the suspension using the trypan blue technique.

A first set of examples concerns the practice of the invention with several procaryotic species. Because bacterial adhesion precedes growth of the bacteria on the surface, the adhesion results will be presented first.

### Example 1

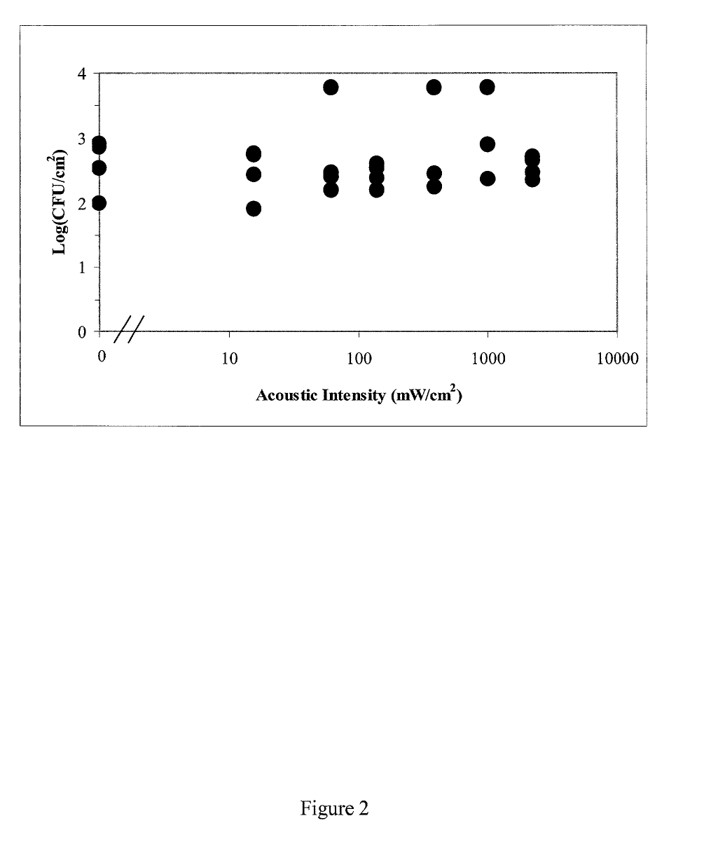

In a first example of the methods of the invention, polyethylene rods were exposed to S. epidermidis according to the methods explained in the methods section above. The results of the one-hour exposure of these polyethylene rods to 10⁵ CFU/ml S. epidermidis are detailed in FIG. 2. FIG. 2 contains a graph showing this data. The x-axis of this graph indicates the various intensities of 70 kHz ultrasound to which the rods were exposed while being incubated with the S. epidermidis. The y-axis indicates the quantity of S. epidermidis found adhered to the rod after the one-hour exposure period.

In this first example, the rods showed similar bacterial adherence under all intensities of ultrasound. The control samples, which were exposed to no ultrasound, showed adherence very similar to that observed with the ultrasound-exposed samples. The points that were not exposed to ultrasound are represented at the Log(I)=0 values on the x-axis.

### Example 2

The initial adherence of bacteria to the rods was shown not to depend on the ultrasonic intensity within the same range examined in Example 1. Subsequent experiments were conducted in which samples were exposed to 2 W/cm² ultrasound and compared to samples not exposed to ultrasound. In these, the concentration of bacteria in the suspension used was varied from 10³ to 10⁵ CFU/ml to evaluate whether initial bacterial concentration would make any difference in the amount of adhesion. For example at a concentration of 10,000 CFU/ml there was an average of 67 CFU/cm² under conditions of incubation, and 71 CFU/cm² under insonation; however, these differences were not found to be statistically significant (p>0.05). No statistically significant differences in bacterial adhesion were observed with or without ultrasound at any of the concentrations used. Thus, without being limited to any one theory, adhesion appears to occur independently of ultrasound at these low frequencies and low power densities.

### Example 3

Since the adhesion experiments appearing above showed that exposure to ultrasound did not inhibit adhesion, another set of experiments was conducted to determine if growth rate was affected by ultrasound. In a first of these experiments, S. epidermidis was grown on similar rods for 6 hours with and without exposure to ultrasound. Following this, the bacteria were stripped from the rods, plated and counted.

In four separate experiments, there was no statistically significant difference in rod-adherent bacteria under the conditions of exposure to ultrasound or simple incubation. These experiments only counted viable bacteria that could be removed from the rods and counted, however.

A follow-up set of experiments measured the amount of bacteria and exopolysaccharide found adherent to the rods after incubation with or without ultrasound using the toluidine blue technique. First, the rods were exposed to the S. epidermidis bacterial suspension for 6 hours (with and without ultrasound) and then stained with toluidine blue.

Following this, the resulting biofilms were dissolved into NaOH. The absorbance of the resulting solution was measured in a spectrophotometer. Three experiments involving eight rods each were performed and the resulting biofilms were measured in the spectrophotometer. The absorbance of the solution obtained from each rod was measured ten times in the spectrophotometer to also assess the precision of the measurements. Three blank samples were also tested in the spectrophotometer. The average absorbance value of the rods exposed to ultrasound was 0.0116 and the average absorbance value of the incubated rods was 0.0112. There was not any statistically significant difference between the ultrasound-exposed and non-ultrasound-exposed sets of rods, however.

### Example 4

In order to enhance the difference in growth in these experiments, glucose was added to the TSB nutrient feeding the S. epidermidis, and experiments similar to Example 3 were repeated with the length of incubation with or without ultrasound extended to 16 hrs. The experiments compared simply incubated rods to those incubated and exposed to 2 W/cm² of 70 kHz ultrasound for 16 hrs. These experiments allowed for more significant growth of the biofilms in the incubated control rods.

The results showed that the rods exposed to the bacteria growing with glucose under ultrasound grew biofilms that were thicker and more uniform. The previous toluidine blue absorbance experiments were designed to test the difference in the amount of biofilm formed when the difference was not visually obvious. However, for bacteria grown in glucose for 16 hours, the difference in the biofilms was visually obvious.

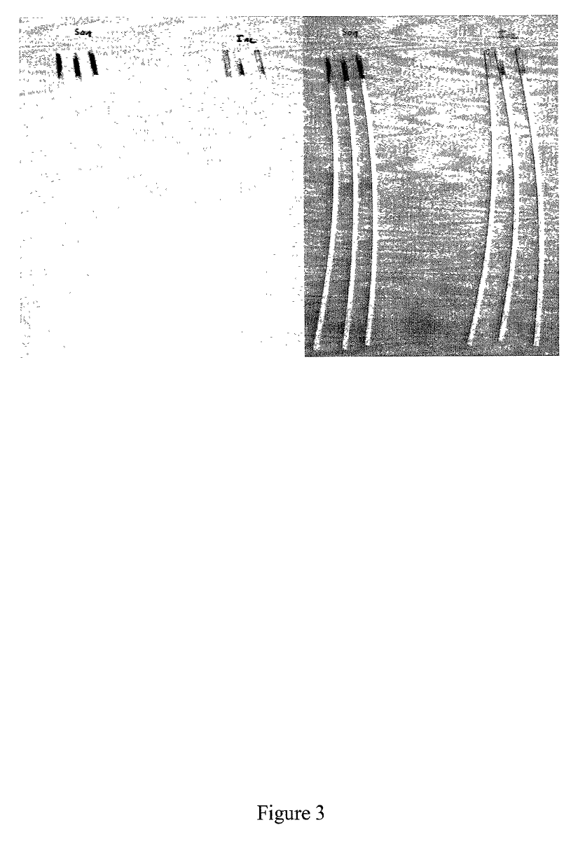

Three experiments using six rods each were performed. Each of these experiments showed significantly more biofilm on the rods incubated under exposure to ultrasound than those incubated without ultrasound. FIGS. 3A, 3B show two photographs of representative toluidine blue-stained biofilms found on the polyethylene rods in two of these experiments following incubation and incubation with ultrasound. In both FIGS. 3A and 3B, the rods exposed to ultrasound are shown on the left of each photograph, and the rods which were incubated are shown on the right of each photograph. In these photographs it can be seen that some S. epidermidis biofilm grew on each of the incubated rods. The biofilms on the insonated rods (those rods exposed to ultrasound), however, are seen to be much more darkly stained and more uniform in appearance over the exposed surface of the rod.

### Example 5

A next experiment was designed to determine whether outside influences could be responsible for causing the incubated rods to grow less biofilm than the rods incubated while being exposed to ultrasound. Two possibilities for experimental artifacts existed that needed to be examined: 1) perhaps a reduced oxygen supply in the cell incubator apparatus (which is a closed box) decreased the rate of bioflhn formation compared to the rods in the ultrasonic bath; 2), the incubated rods were swirled at 100 rpm (to ensure good transport of oxygen), but perhaps the swirling was inhibiting good biofilm growth. These two possibilities were tested by adding two control groups to the experiment. The first possibility was tested by placing another group of rods in the constant temperature water bath that was supplying the water to the ultrasonic bath, which was at 37° C., and outside of both the incubator apparatus and the ultrasound bath. The second possibility was examined by placing one group of rods in the incubator, but this group was not placed on the orbital shaker. These two control groups allowed analysis of the effects of the incubator's atmosphere and shaker upon the experiments.

The results of these experiments showed that all of the above groups grown without ultrasound grew similar biofilms, yet the rods exposed to ultrasound grew visually thicker biofilms. These results indicate that neither shaking nor the enclosed incubator apparatus inhibits biofilm growth. The validation that experimental artifacts do not arise from these procedures improves the reliability of the results. This indicates that the results of the earlier examples showing that ultrasound enhances biofilm formation support that theory.

Since earlier experiments showed that ultrasound could enhance biofilm formation by S. epidermidis, similar experiments were conducted using two other bacterial species to evaluate whether the phenomenon was species independent. Specifically, E. coli and P. aeruginosa were used as the procaryotic species tested in the next experiments.

### Example 6

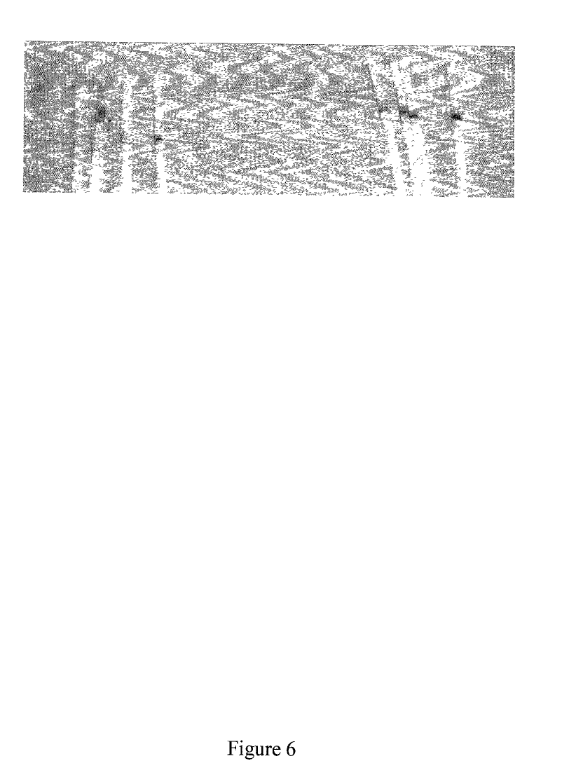

The experiments with E. coli were conducted for a duration of 24 hours to allow sufficient biofilm formation. This was done because E. coli does not generally form biofilms as quickly as S. epidermidis RP62A. Experiments using E. coli were also repeated three times. These experiments also showed a significant increase in biofilm formation for the biofilms grown in the presence of 2 W/cm² 70 kHz ultrasound. The results of this Example are illustrated in FIG. 6. The dark rings on each rod are not biofilm but are stained dried tryptic soy broth that dried at the interface between the air and the bacterial culture during the experiment. The biofilm occupies the area between the blue rings and the end of the rod, and was much more visible on the insonated rods (on the left of FIG. 6) than on the rods not exposed to ultrasound (on the right).

The P. aeruginosa experiments were extended to 48 hours in duration to allow sufficient biofilm formation. This was done since P. aeruginosa biofilms grow slower than either of the other two species. The rods exposed to ultrasound were also exposed to 2 W/cm² 70 kHz ultrasound, but the ultrasound was pulsed in a 1:5 duty cycle. Ultrasound was delivered in a 100 millisecond pulse of 70 kHz ultrasound, and the pulse was repeated each 500 milliseconds for 48 hrs.

The stained rods were not as visually disparate as the bacterial biofilms observed with the other procaryotic organisms tested, yet some biofilm could be seen on some of the rods incubated with ultrasound, while no biofilm could be observed on any of the incubated rods. The amount of bacteria and exopolysaccharide on the rods was quantified using the aforementioned spectrophotometric technique. The results of this experiment are shown in FIG. 3.

FIG. 3 shows the absorbance observed from stained 48-hour P. aeruginosa biofilms from the polyethylene rods. Data are shown from two rounds of the experiment. In a first, the rods were exposed to 1:5 pulsed 2.2 W/cm² 70 kHz ultrasound, and in a second, the rods were exposed to 1:5 pulsed 1.5 W/cm² 70 kHz ultrasound. These data points are compared with those exposed to no ultrasound, here seen distributed along the y-axis.

Statistical tests (Student-t comparison of means) determined that the rods incubated with exposure to ultrasound had more stain from biofilm than the control rods at the 0.1 level of significance. It should also be noted that the averages of the samples increased with increasing ultrasound, and while large variations existed within the group exposed to ultrasound, all of the values but one were larger than those of the incubated group. These results appear to indicate that P. aeruginosa biofilm growth is also accelerated by ultrasound.

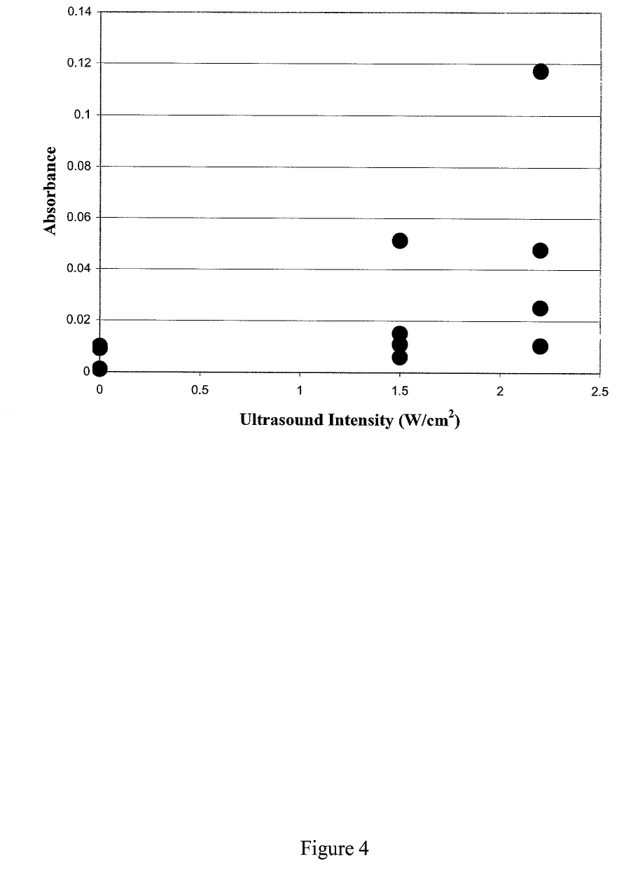

Planktonic cultures of bacteria showed normal growth during the experiments, but consistently more growth was observed when exposed to ultrasound than when incubated without ultrasound. FIG. 4 shows the mean and 95% confidence intervals from four replicates of S. epidermidis growing in TSB. Half of the test tubes were incubated while the others were exposed to 3 W/cm² ultrasound at 70 kHz. Experiments with the other two species also showed enhanced planktonic growth under ultrasonic exposure. For example, E. coli after growing 3 hours in 70 kHz ultrasound had an average planktonic concentration of 8.5×10⁷ CFU/ml, whereas without ultrasound the average concentration was 4.8×10⁷ CFU/ml. These differences are statistically significant (n=4, p=0.050). Likewise for P. aeruginosa there was an average planktonic concentration of 4.8×10∂CFU/ml after 3 hours of insonation, whereas without planktonic concentration of 3.5×10⁷ CFU/ml after 3 hours. These differences are also statistically significant (n=0, p=0.047).

### Example 7

Having tested the method of the invention on multiple procaryotic species, experiments were designed to evaluate the utility of the methods of the invention when used with eucaryotic cell cultures. In a first round of experiments, HeLa cells were used. In a first iteration of the HeLa experiments, four T-25 filter top flasks were seeded at 30,000 HeLa cells/ml. The flasks were labeled and allowed to sit in an incubator for 24 hours. The flasks were then placed in their respective positions in separate Sonicor baths under conditions of: ˜5% CO₂, 37° C.

After 24 hours of incubation, ultrasound was applied to one bath at 18 mW/cm² for 72 hrs. The flasks exposed to ultrasound had an average of 1.72×10⁵ cell/ml in suspension, whereas the incubated flasks had an average of 1.51×10⁵ cell/ml. On the surface of the flask exposed to ultrasound, the concentration of cells was 0.42×10⁵ cell/cm² whereas the incubated flask had 0.37×10⁵ cell/cm².

In another iteration of the experiment, four T-25 filter top flasks were seeded at 57,000 HeLa cells/ml. The flasks were labeled and allowed to sit in an incubator for 24 hours. Then they were placed in their respective positions in Sonicor baths under conditions of: ˜5% CO₂, 37° C. After 24 hours, the ultrasound was applied in one bath at 8 mW/cm² for 72 hrs. The ultrasonicated flasks (i.e., those exposed to the ultrasound) had an average of 4.25×10⁵ cell/ml in suspension whereas the incubated flasks had an average of 1.95×10⁵ cell/ml. On the surface, the flask exposed to ultrasound had 0.68×10 cell/cm² whereas the incubated flask had 0.65×10⁵ cell/cm².

In still another iteration of the experiment, four T-25 filter top flasks were seeded at 30,000 HeLa cells/ml. The flasks were labeled and allowed to sit in an incubator for 24 hours. Then they were placed in their respective positions in Sonicor baths under conditions of: ˜5% CO₂, 37° C. After 24 hours, ultrasound was applied in one bath at 12 mW/cm² for 72 hrs. The flasks exposed to ultrasound had an average of 2.25×10⁵ cell/ml in suspension whereas the incubated flasks had an average of 0.64×10⁵ cell/ml. On the surface the flask exposed to ultrasound had 0.31×10⁵ cell/cm² whereas the incubated flask had 0.75×10⁵ cell/cm².

In yet another iteration of the experiment of the invention, four T-25 filter top flasks were seeded at 43,000 HeLa cells/ml. The flasks were labeled and allowed to sit in an incubator for 24 hours. They were then placed in their respective positions in Sonicor baths under conditions of: ˜5% CO₂, 37° C. After 24 hours, ultrasound was applied in one bath at 15 mW/cm² for 72 hrs. The flasks exposed to ultrasound had an average of 3.42×10⁵ cell/ml in suspension whereas the incubated flasks had an average of 0.21×10⁵ cell/ml. On the surface, the flask exposed to ultrasound had 0.31×10⁵ cell/cm² whereas the incubated flask had 1.98×10⁵ cell/cm².

Four T-25 regular top flasks were next seeded at 71,000 HeLa cells/ml. The flasks were labeled and allowed to sit in an incubator for 24 hours. The flasks were then placed in their respective positions in Sonicor baths under conditions of: ˜5% CO₂, 37° C. After 24 hours, ultrasound was applied in one bath at 10 mW/cm² for 24 hrs. The flasks exposed to ultrasound had an average of 0.95×10⁵ cell/ml in suspension whereas the incubated flasks had an average of 0.96×10⁵ cell/ml. On the surface the flask exposed to ultrasound had 0.72×10⁵ cell/cm² whereas the incubated flask had 0.60×10⁵ cell/cm².

In a final iteration, four T-25 filter top flasks were seeded at 57,000 HeLa cells/ml. These flasks were then labeled and allowed to sit in an incubator for 24 hours. Next they were placed in their respective positions in Sonicor baths under conditions of: ˜5% CO₂, 37° C. After 24 hours, the ultrasound was applied in one bath at 8 mW/cm² for 24 hrs. The flasks exposed to ultrasound had an average of 2.93×10⁵ cell/ml in suspension, whereas the incubated flasks had an average of 3.60×10⁵ cell/ml. On the surface the ultrasonicated flask had 0.47×10⁵ cell/cm² whereas the incubated flask had 0.58×10⁵ cell/cm². Thus, in regard to HeLa cells, it appears that ultrasonic treatment does increase the amount of cells present in a sample compared to the number present in a control, particularly when insonation is applied for 72 hours.

### Example 8

WiDR cells were used in a next set of eucaryotic experiments to assure that the methods of the invention have utility in multiple eucaryotic species. In a first iteration of the experiment, four T-25 filter top flasks were seeded at 14,000 WiDR cells/ml. The flasks were labeled and allowed to sit in an incubator for 24 hours. The flasks were then placed in Sonicor baths under conditions of: ˜5% CO₂, 37° C. After 24 hours, ultrasound was applied in one bath at 10 mW/cm² for 24 hrs. The flasks exposed to ultrasound had an average of 4.13×10⁵ cell/ml in suspension whereas the incubated flasks had an average of 3.85×10⁵ cell/ml. On the surface the flask exposed to ultrasound had 0.044×10⁵ cell/cm² whereas the incubated flask had 0.042×10⁵ cell/cm².

In a second iteration of the experiment, four T-25 filter top flasks were seeded at 14,000 WiDR cells/ml. The flasks were labeled and allowed to sit in an incubator for 24 hours. The flasks were then placed in Sonicor baths under conditions of: ˜5% CO₂, 37° C. After 24 hours, ultrasound was applied in one bath at 10 mW/cm² for 24 hrs. The flasks exposed to ultrasound had an average of 3.06×10⁵ cell/ml in suspension, whereas the incubated flasks had an average of 2.58×10⁵ cell/ml. On the surface, the flask exposed to ultrasound had 0.081×10⁵ cell/cm² whereas the incubated flask had 0.139×10⁵ cell/cm².

### Example 9

The method of the invention was next tested using TK-6 cells, which are considered anchorage independent. In this example, four T-25 filter top flasks were seeded at 71,000 TK-6 cells/ml. The flasks were labeled and allowed to sit in an incubator for 24 hours. The flasks were then placed in Sonicor baths under conditions of: ˜5% CO₂, 37° C. After 24 hours, ultrasound was applied in one bath at 8 mW/cm² for 24 hrs. The flasks exposed to ultrasound had an average of 13.9×10⁵ cell/ml in suspension whereas the incubated flasks had an average of 9.1×10⁵ cell/ml.

In summary for eucaryotic cells, the application of low frequency, low intensity ultrasound increases their growth. This was particularly the case in regard to cells in suspension. In some experiments the concentration of cells on the surface increased, but not to the extent as cells in suspension. Ultrasonic treatment of HeLa cells, for example, for 72 hours appears to increase the difference in the amount of cells (compared to non-treated cells) more than does treatment for 24 hours. Within the narrow range of power densities examined, there does not appear to be a correlation between intensity and cell growth. Without being limited to any one theory, it appears that ultrasound may remove some cells from the surface into the media above the surface. Further, an optimum intensity window may exist wherein ultrasound enhances growth more than removal, thus increasing the net amount of cells on a surface. In summary for procaryotic cells, the application of low frequency, higher intensity ultrasound increases their net growth both on surfaces and in suspension.

### SUMMARY

The invention provides methods of enhancing the growth rate of cells in culture by exposing them to ultrasound of specified frequencies and intensities during incubation. These methods are beneficial with both eucaryotic and procaryotic cells, and operate with cells grown in planktonic suspension and with cells grown on substrates such as biofilms. This exposure to ultrasound encourages cell growth by increasing convection in the cell growing medium, thus allowing better oxygen and nutrient uptake, as well as by more rapidly removing cell waste products. In regard to biofilms, ultrasound renders them more permeable to small molecule flow, thus reducing nutrient gradients previously observed in such films.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed and desired to be secured by United States Letters Patent is:

1. A method of increasing the growth rate of a cell in culture comprising exposing the cell to ultrasound of a selected frequency and intensity.

2. The method of claim 1, wherein the frequency of the ultrasound is from about 20 kHz to about 1 MHz.

3. The method of claim 2, wherein the frequency of the ultrasound is from about 20 kHz to about 100 kHz.

4. The method of claim 3, wherein the frequency of the ultrasound is about 70 kHz.

5. The method of claim 1, wherein the intensity of the ultrasound is from about 1 mW/cm² to about 5 W/cm².

6. The method of claim 5, wherein the intensity of the ultrasound is from about 1 W/cm² to about 3 W/cm².

7. The method of claim 5, wherein the intensity of the ultrasound is from about 1 mW/cm² to about 50 mW/cm².

8. The method of claim 7, wherein the intensity of the ultrasound is from about 8 mW/cm² to about 18 mW/cm².

9. A method of increasing the growth rate of a procaryotic cell in culture comprising exposing the cell to ultrasound of a selected frequency and intensity.

10. The method of claim 9, wherein the frequency of the ultrasound is from about 20 kHz to about 1 MHz.

11. The method of claim 9, wherein the frequency of the ultrasound is from about 20 kHz to about 100 kHz.

12. The method of claim 9, wherein the frequency of the ultrasound is about 70 kHz.

13. The method of claim 9, wherein the intensity of the ultrasound is from about 1 mW/cm² to about 5 W/cm².

14. The method of claim 9, wherein the intensity of the ultrasound is from about 1.5 W/cm² to about 2.5 W/cm².

15. The method of claim 9, wherein the intensity of the ultrasound is from about 2 W/cm ²to about 2.2 W/cm².

16. The method of claim 9, wherein the procaryotic cells are selected from the group consisting of: Staphylococcus epidermidis, Pseudomonas aeruginosa, and Escherichia coli.

17. A method of increasing the growth rate of a eucaryotic cell in culture comprising exposing the cell to ultrasound of a selected frequency and intensity.

18. The method of claim 17, wherein the frequency of the ultrasound is from about 20 kHz to about 1 MHz.

19. The method of claim 17, wherein the frequency of the ultrasound is from about 20 kHz to about 100 kHz.

20. The method of claim 17, wherein the frequency of the ultrasound is about 70 kHz.

21. The method of claim 17, wherein the intensity of the ultrasound is from about 1 W/cm² to about 1 W/cm².

22. The method of claim 17, wherein the intensity of the ultrasound is from about 8 mW/cm² to about 50 mW/cm².

23. The method of claim 17, wherein the intensity of the ultrasound is from about 10 mW/cm² to about 18 mW/cm².

24. The method of claim 17, wherein the eucaryotic cells are selected from the group consisting of: HeLa cells, WiDR cells, and TK-6 cells.

25. A method of increasing the growth rate of a biofilm in culture comprising exposing the biofilm to ultrasound having a frequency of from about 20 kHz to about 1 MHz, and having an intensity of from about 1 mW/cm² to about 5 W/cm².

26. The method of claim 25, wherein the frequency of the ultrasound is from about 20 kHz to about 100 kHz.

27. The method of claim 25, wherein the frequency of the ultrasound is about 70 kHz.

28. The method of claim 25, wherein the intensity of the ultrasound is from about 1 W/cm² to about 3 W/cm².

29. The method of claim 28, wherein the intensity of the ultrasound is from about 2 W/cm² to about 2.5 W/cm².

30. The method of claim 25, wherein the intensity of the ultrasound is from about 8 mW/cm² to about 50 mW/cm².

31. The method of claim 30, wherein the intensity of the ultrasound is from about 10 mW/cm² to about 18 mW/cm².

32. A method of increasing the growth rate of a planktonic cell culture comprising exposing the planktonic cell culture to ultrasound having a frequency of from about 20 kHz to about 1 MHz, and having an intensity of from about 1 mW/cm² to about 5 W/cm².

33. The method of claim 32, wherein the frequency of the ultrasound is from about 20 kHz to about 100 kHz.

34. The method of claim 33, wherein the frequency of the ultrasound is about 70 kHz.

35. The method of claim 32, wherein the intensity of the ultrasound is from about 1 W/cm² to about 3 W/cm².

36. The method of claim 35, wherein the intensity of the ultrasound is from about 2 W/cm² to about 2.2 W/cm².

37. The method of claim 32, wherein the intensity of the ultrasound is from about 8 mW/cm² to about 50 mW/cm².

38. The method of claim 37, wherein the intensity of the ultrasound is from about 10 mW/cm² to about 18 mW/cm².
